(19) United States
(12) Patent Application Publication
(10) Pub. No.: US 2020/0383612 A1
(43) Pub. Date: Dec. 10, 2020

(54) SYSTEMS AND METHODS FOR INFECTION PREVENTION

(71) Applicant: Barry Hunt, Cambridge (CA)

(72) Inventor: Barry Hunt, Cambridge (CA)

(21) Appl. No.: 16/868,687

(22) Filed: May 7, 2020

Related U.S. Application Data
(60) Provisional application No. 62/844,495, filed on May 7, 2019.

(51) Int. Cl. A61B 5/11; A61B 5/00; A61B 5/01

(52) U.S. Cl. CPC A61B 5/1128; A61B 5/0013; A61B 2562/0271; A61B 5/7246; A61B 5/015; A61B 5/1113

(57) ABSTRACT

Systems and methods of tracking interactions of a person in a facility are described herein. The methods include capturing, by at least one sensor positioned in the facility, thermal infrared image data; transmitting the thermal infrared image data to a receiver for processing the thermal infrared image data; detecting motion of at least one person in the facility by comparing pixels or regions around the pixels of consecutive image frames of thermal infrared image data received from the at least one sensor; and when motion of at least one person in the facility is detected, comparing the motion of the at least one person in the facility to an object in the facility to predict if the person interacted with the object in the facility.

### CROSS-REFERENCE TO RELATED APPLICATIONS

This application claims priority to U.S. Provisional Patent Application No. 62/844,495 that was filed on 7 May 2019, the contents of which are incorporated herein by reference for all purposes.

### TECHNICAL FIELD

The embodiments disclosed herein relate to systems and methods for infection prevention, and in particular to systems and methods for infection prevention in a facility by tracking hygiene compliance of persons in the facility.

### BACKGROUND

Medical facilities such as hospitals, clinics, nursing homes and the like have seen increased occurrences of multi-resistant highly infectious and virulent super bugs, such as Methicillin Resistant Staphylococcus Aureus (MRSA). Medical facility-acquired infections are incredibly costly to the healthcare system as these types of infections generally take longer to treat than infections acquired in a community-setting. Medical facility-acquired infections can also be more dangerous to patients than infections acquired in a community as patients that contract these diseases when in the medical facility itself typically have an immune system that is already in a weakened state. Accordingly, these diseases, pose a significant threat to the provision of safe and effective health care treatment to patients.

Poor personal hygiene, including inadequate handwashing, and ineffective sterilization of surfaces within the hospital are some of the factors that lead to the spread of hospital-acquired infections. For example, it is widely known that spread of the MRSA bug in hospitals and similar facilities is predominantly through direct contact between patients and their care givers. To eradicate facility-acquired infections it is therefore important for facilities to track movement of and interactions between patients and care givers.

Systems that have been developed to track and analyze human interactions in a clinical setting have focused primarily on single modality sensing. For example, Radio Frequency Identification (RFID), infrared (IR), manual key input, written bed board updates or human observatory monitoring schemes are traditionally used to record human interactions in a clinical setting.

In one specific example, RFID-based systems have been developed to track individual movements to identify human activities that occur within the healthcare facility. Typically, RFID-based sensor systems take the form of location and contact make/break sensing systems for certain protocol adherence. As one example, an institution may specify that staff members shall sanitize their hands upon entrance into the patient's room. An RFID-based sensor system may determine if a staff member was in the presence of a hand sanitization station, or if cleansing agents are dispensed, and a process defect may be alarmed or recorded when staff do not appear to have sanitized their hands. Accordingly, typical RFID-based systems produce a discrete snapshot in time of positions of individuals within the facility and do not provide contextual information over a period of time.

Thermography has recently emerged as a new mechanism for tracking movement of individuals. Thermographic cameras detect radiation in the long-infrared range of the electromagnetic spectrum (roughly 9,000-14,000 nanometers or 9-14 μm) and produce images of that radiation, called thermograms. Since infrared radiation is emitted by all objects with a temperature above absolute zero, thermography makes it possible to see one's environment with or without visible illumination and without receiving any identifiable information. The amount of radiation emitted by an object increases with temperature; therefore, thermography allows one to see variations in temperature. When viewed through a thermal imaging camera, warm objects stand out well against cooler backgrounds and humans and other warm-blooded animals become easily visible against the environment, day or night.

Machine vision refers to technologies that can be used to provide imaging-based automatic inspection and analysis for such applications as automatic inspection, process control, and robot guidance. Machine vision is a term encompassing a large number of technologies, software and hardware products, integrated systems, actions, methods and expertise.

There is a need in the field for improvements in the development of systems for tracking human movement in a facility and for analyzing human interactions in a healthcare facility.

### SUMMARY

According to some embodiments, a method of tracking interactions of a person in a facility is described herein. The method includes capturing, by at least one sensor positioned in the facility, thermal infrared image data; transmitting the thermal infrared image data to a receiver for processing the thermal infrared image data; detecting motion of at least one person in the facility by comparing pixels or regions around the pixels of consecutive image frames of thermal infrared image data received from the at least one sensor; and when motion of at least one person in the facility is detected, comparing the motion of the at least one person in the facility to an object in the facility to predict if the person interacted with the object in the facility.

According to some embodiments, before the detecting motion of at least one person in the facility, the method includes isolating thermal infrared person data representing the position of the person in the facility from the thermal infrared image data.

According to some embodiments, the isolating thermal infrared person data includes performing edge detection on the thermal infrared image data to identify the thermal infrared person data of the thermal infrared image data.

According to some embodiments, the method also includes determining a route of the person through the facility based on the motion of the person in the facility over time.

According to some embodiments, the method also includes determining a likelihood that the person is a health care professional based on the route of the person through the facility.

According to some embodiments, the objects are personal hygiene devices, the at least one person in the facility is a health care worker, and the method further includes creating a hygiene standards compliance profile based on determining if the health care worker interacted with the personal hygiene devices in the facility; and forwarding the hygiene standards compliance profile to a third party service provider.

According to some embodiments, a system for tracking interactions of a person in a facility is described herein. The system includes at least one processor configured to receive thermal infrared image data from at least one sensor positioned in the facility; detect motion of at least one person in the facility by comparing pixels or regions around the pixels of consecutive image frames of thermal infrared image data received from the at least one sensor; and, when motion of at least one person in the facility is detected, compare the motion of the at least one person in the facility to an object in the facility to predict if the person interacted with the object in the facility.

According to some embodiments, the processor is further configured to, before detecting motion of at least one person in the facility, isolate thermal infrared person data representing the position of the person in the facility from the thermal infrared image data.

According to some embodiments, the processor is further configured to isolate the thermal infrared person data by performing edge detection on the thermal infrared image data to identify the thermal infrared person data of the thermal infrared image data.

According to some embodiments, the processor is further configured to determine a route of the person through the facility based on the motion of the person in the facility over time.

According to some embodiments, the processor is further configured to determine a likelihood that the person is a health care professional based on the route of the person through the facility.

According to some embodiments, the objects are personal hygiene devices, the at least one person in the facility is a health care worker, and the processor is further configured to create a hygiene standards compliance profile based on determining if the health care worker interacted with the personal hygiene devices in the facility; and forward the hygiene standards compliance profile to a third party service provider.

Other aspects and features will become apparent, to those ordinarily skilled in the art, upon review of the following description of some exemplary embodiments.

### BRIEF DESCRIPTION OF THE DRAWINGS

The drawings included herewith are for illustrating various examples of articles, methods, and apparatuses of the present specification. In the drawings:

FIG. 1 is a block diagram of an infection prevention system, according to one embodiment;

FIG. 2 is a block diagram showing positioning a plurality of sensors and their respective regions within a facility, according to one embodiment;

FIG. 3 is a block diagram showing components of a server of the system of FIG. 1, according to one embodiment;

FIG. 4 is a block diagram showing communication between a receiver of FIG. 1 and a plurality of service providers, according to one embodiment; and

FIG. 5 is a block diagram showing a method of tracking a person of interest in a facility, according to one embodiment.

### DETAILED DESCRIPTION

Various systems and processes will be described below to provide an example of each claimed embodiment. No embodiment described below limits any claimed embodiment and any claimed embodiment may cover systems or processes that differ from those described below. The claimed embodiments are not limited to systems or processes having all of the features of any one system or process described below or to features common to multiple or all of the systems or processes described below.

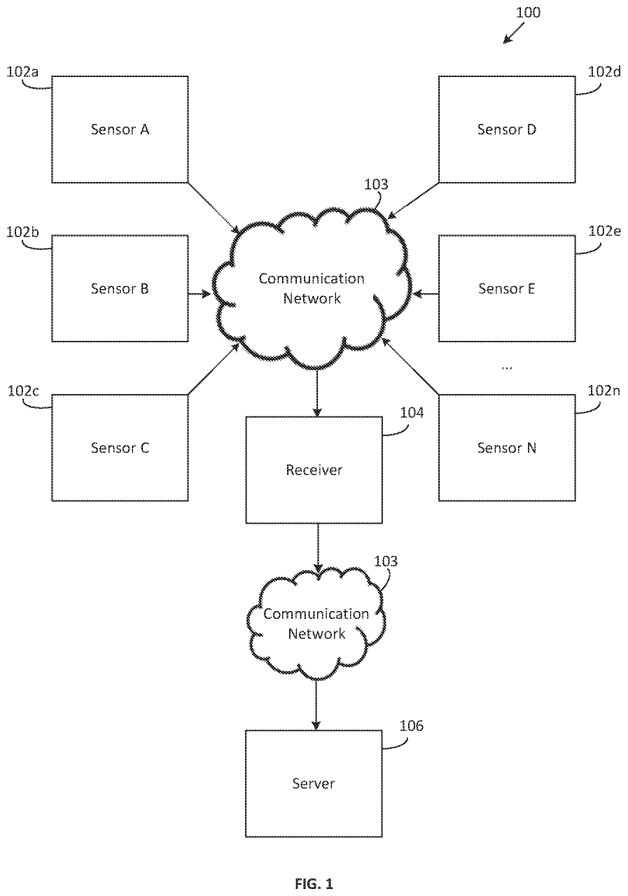

Referring now generally to FIG. 1, illustrated therein is a tracking system 100 for a facility, according to one exemplary embodiment. The tracking system 100 generally includes a plurality of sensors 102 (i.e. sensors 102a, 102b, 102c, 102d, 102e, . . . , 102n) disposed within the facility, a communication network 103, a receiver 104 and a server 106.

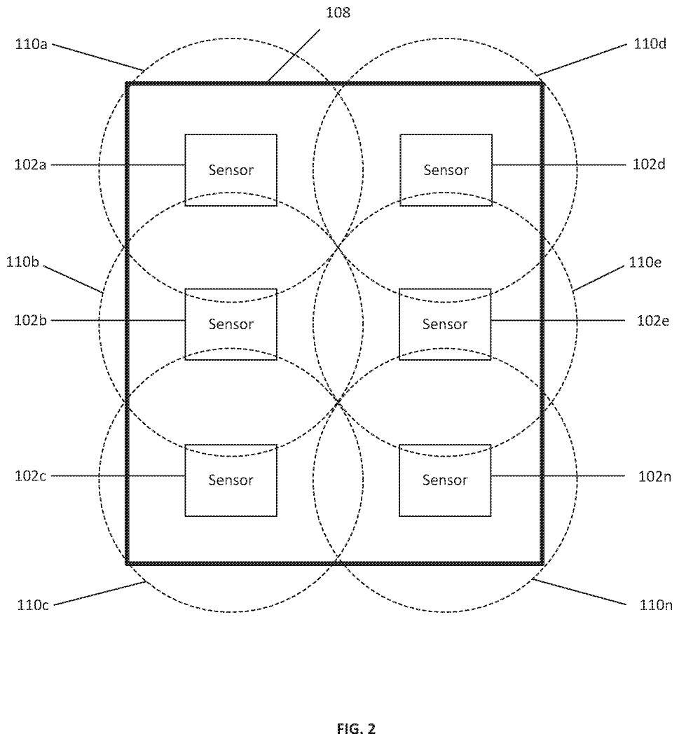

As shown in FIG. 2, sensors 102 generally detect movement within the facility 108, such as but not limited to movement of individuals within the facility 108. Sensors 102 can be any appropriate sensor for detecting movement of individuals within the facility 108. For instance, in some embodiments, the sensors 102 may generally measure thermal infrared radiation (e.g. as pixel data) emitted in a respective region 110 (i.e. regions 110a, 110b, 110c, 110d, 110e, . . . , 110n) of a facility 108. The thermal infrared radiation is typically emitted by objects (e.g. machinery, people, etc.) in the mid-infrared thermal band (i.e., 3 microns to 5 microns band) and far-infrared thermal band (i.e., 8 microns to 14 microns band) of the electromagnetic spectrum. For instance, sensors 102 may detect temperature changes in regions 110 caused by human emissions in the mid-infrared thermal band and far-infrared thermal band of the electromagnetic spectrum. Slightly shorter or slightly longer ranges may also yield acceptable detection results.

In these embodiments, sensors 102 may include one or more thermal cameras having pixel arrays sensitive to the mid-infrared and/or far-infrared bands of the electromagnetic spectrum. For example, in one embodiment, sensors 102 may be a thermal activity sensors (TASs) manufactured by ULIS, a manufacturer of thermal sensors. TASs are thermal activity sensors that run entirely on battery power. TASs can provide 80×80 pixels thermal activity sensors and transmit occupancy rates at regular intervals (approximately every two minutes), without compromising the privacy of the occupants. In another embodiment, sensors 102 may be thermal activity sensors that do not run on battery power.

In other embodiments, the sensors 102 may include cameras that collect images from a facility 108 where individuals are located. Output from the cameras is directed into a computer where it is processed.

In other embodiments, sensors 102 may be motion detection sensors such as but not limited to optical sensors, pressure sensors or the like. For instance, sensors 102 may be positioned in a bed, a chair or in any other position where they may detect a movement of an individual within the facility 108.

Facility 108 can include any number of sensors 102 arranged in any pattern to detect the presence and/or movement of individuals within the facility 108. In some embodiments, sensors 102 may be arranged within facility 108 such that an area 109 where the presence and/or movement of individuals is detectable by sensors 102, the area 109 comprising regions 110, covers the entirety of facility 108. In some embodiments, neighboring regions 110 may overlap to provide for detecting the presence and/or movement of individuals within an entire area of facility 108.

Regions 110 can be any appropriate shape and size. In the embodiment shown in FIG. 2, regions 110 are shown as being approximately circular in shape. The shape and size of regions 110 can be defined by sensors 102 or receiver 104 (e.g. software running on receiver 104). In some embodiments, each sensor 102 may detect mid-infrared and/or far-infrared bands of the electromagnetic spectrum for a region approximately 30 square meters in size. In some embodiments, facility 108 can represent a traditional physical facility (e.g. a hospital) or a portion of a traditional physical facility (e.g. a patient room within a hospital).

Thermal infrared radiation measured by sensors 102 of objects in regions 110 is suitable to indicate temperature variations present within regions 110. Thermal infrared radiation measurements can be stored as pixel data by sensors 102 and used to produce a thermogram of the area of region 110 corresponding to each respective sensor 102. For example, sensor 102a can produce a thermogram of infrared radiation measurements for region 110a.

In one embodiment, thermograms can be produced by sensors 102 that are representative of local temperature changes in regions 110 of facility 108 caused by the movement of humans within facility 108. Thermograms from various sensors 102 are produced over time and can be combined and analyzed together to track changes in thermographic radiation in the facility 108 over time.

In one specific embodiment, changes in thermographic radiation over time within facility 108 can represent the entrance, movement within (i.e. change in position) and exit of a person through facility 108. For example, changes in temperature in regions 110 of facility 108 that are caused by the presence of a person, and with suitable monitoring of emissions from the person in the thermal infrared spectrum over time within regions 110 of facility 108, detection of the movement of the person between regions 110 of the facility 108 can be achieved.

Returning to FIG. 1, in some embodiments, sensors 102 each include a transmitter (not shown). After measuring thermal infrared radiation and storing pixel data derived therefrom emitted within a respective region 110 as a thermogram, each sensor 102 provides the thermal infrared image data to a receiver 104 via its transmitter over a communication network 103.

Receiver 104 receives the thermal infrared image data from each of the sensors 102 in facility 108 via communication network 103. Receiver 104 comprises a computer system and software components stored in a memory. Receiver 104 may or may not be physically positioned in the facility 108. Receiver 104 may store the thermal infrared image data in storage (not shown). Receiver 104 collects the thermal infrared image data from sensors 102, optionally processes the thermal infrared image data and transmits the thermal infrared image data to a server 106, also over communication network 103. In some embodiments, data processing at receiver 104 can be in the same manner as described below with respect to server 106.

Receiver 104 and/or server 106 may include one or more modules configured to provide operative control to sensors 102. The modules include, for example, hardware, software, or a combination of hardware and software configured to achieve the desired function of each module. As an illustrative example, each module can include hardware (e.g., electrical circuit components, displays, sensors, etc.) and software (e.g., functions, subroutines, executable programs, etc.) associated with the functional and operative control of the module.

It should be noted that two exemplary communications networks 103 are shown in FIG. 1. The two communication networks 103 shown in FIG. 1 can represent a single communication network 103, two individual communication networks 103 of the same type (e.g. WiFi networks) or two individual communication networks 103 of different types (e.g. a Bluetooth® network and a WiFi network). Exemplary communication networks 103 include local area networks (LANs) and/or wide area networks (WANs). Such networking environments are commonplace in offices, enterprise-wide computer networks, intranets, and the Internet. Further, communication networks 103 may include other forms of wireless communication including but not limited to RFID and Bluetooth. For instance, each sensor 102 may transmit thermal infrared image data to receiver 104 over one type of communication network (e.g. Bluetooth) and receiver 104 may transmit the thermal infrared image data to server 106 over another type of communication network (e.g. Internet).

When utilized in a WAN networking environment, receiver 104 might comprise a modem or other means for establishing communications over the WAN, such as the Internet. It will be appreciated by those of ordinary skill in the art that the network connections shown are exemplary and other means of establishing a communications link between each sensor 102 and the receiver 104 and between the receiver 104 and the server 106 might be utilized.

In use, each sensor 102 transmits a signal to the receiver 104 over communication network 103. The signal may be periodically (e.g. at set time intervals) transmitted by each sensor 102 using the co-ordinates of the receiver 104 in the facility 108 at that point in time or, alternatively, the signal may be transmitted upon sensing movement of a person within a region 110 (e.g. a change in the thermogram of the facility). Further, the detection data (e.g. thermographic data) may be temporarily stored at the and periodically (e.g. at set time intervals) transmitted by receiver 104 to server or, alternatively, the thermographic data may be transmitted by receiver 104 to server 106 upon receipt from sensors 102 (e.g. upon sensors 102 sensing movement of a person within a region 110 of facility 108).

In one embodiment, each region 110 can be associated with one or more specific physical structures in facility 108, such as but not limited to sanitation devices (e.g. a sink unit, an antibacterial fluid dispenser or another similar sanitation device). Each sensor 102 can therefore indicate when a person has traveled to and/or spent time in proximity to a particular physical structure such as but not limited to a specific sanitation device and therefore can indicate whether or not a disinfection procedure (e.g. handwashing) has been carried out.

In addition to indicating whether or not a hand washing procedure has been carried out, a signal transmitted from the sensor 102 to receiver 104 may provide co-ordinates of the physical structure in the medical facility and the time of disinfection procedure (e.g. hand washing) occurring. This may provide for a hygiene standards compliance profile to be generated for the physical structure. The hygiene standards compliance profile may simply be that a particular physical structure is used a certain number of times in a predetermined time period or that a particular physical structure is used every time that a person enters a particular facility or region of a facility. The information conveyed will largely depend on the complexity of the identification signal transmitted and furthermore will depend on the capability of the mobile network unit itself e.g. GPS capability.

In one exemplary embodiment, facility 108 can represent a patient room in a hospital and sensors 102 can be installed in the patient room and used to track the movement of health care professionals (e.g. nurses, physicians, other health care workers) in, out and within the patient room. In one specific embodiment, the sensors 102 can be installed in a patient room of a hospital and are used to track the movement of health care professionals that come in contact with patients in the patient room to agnostically assess hygiene compliance of healthcare workers in the patient room. In this example, each sensor 102 can be positioned on a ceiling of the patient room, for example, and equally spaced from each other sensor 102 to provide for at least one sensor to detect thermographic radiation emitted from persons in all areas of the patient room. In this situation, detection is performed to determine in which regions 110 of the room a person is present, in which regions 110 of the room persons enter and to compare the regions of the room that persons enter to locations of sanitation devices to assess the hygiene compliance of persons in the patient room.

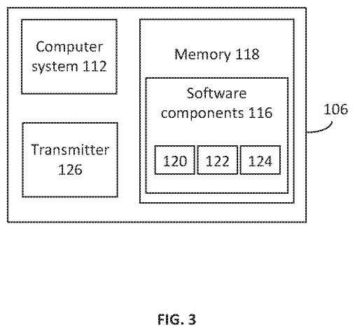

Referring now to FIG. 3, one embodiment of server 106 is shown therein. Server 106 comprises computer system 112 and software components 116 stored in a memory 118. Server 106 may or may not be physically positioned in the facility 108.

Although the following processing of thermal infrared data is described as being performed at the server 106, It should be noted that in some embodiments processing of thermal infrared data may be performed by the receiver 104. In these embodiments, receiver 104 comprises analogous components as are described below with respect to server 106 and processing of the thermal infrared data is performed by the receiver 104 in the same manner as described below with respect to the server 106.

In some embodiments, server 106 includes a computer system 112 operable to execute software to provide for tracking the movement of the person based on thermal infrared image data. Although server 106 may be implemented using software executable using a computer apparatus, other specialized hardware may also provide for tracking the movement of the person based on thermal infrared image data.

The computer system 112 may be, for example, any fixed or mobile computer system, e.g., a personal computer or a minicomputer. The exact configuration of the computer system 112 is not limiting and most any device capable of providing suitable computing capabilities may be used. Further, various peripheral devices, such as a computer display, a mouse, a keyboard, memory, a printer, etc., are contemplated to be used in combination with a processing apparatus in the computer system 112.

Server 106 may also include software components 116 for analysis of the thermal infrared image data received from each of the sensors 102 via receiver 104. One or more of such software components 116 may be used to operate on the thermal image data (e.g. pixel data) provided from the sensors 102 to determine if an individual is within a region 110. Such algorithmic software components 116 for analysis of the thermal image data are shown as a part of an exemplary block diagram of server 106 in FIG. 3.

In operation, an organization might enter commands and information into the computer system 112 or convey the commands and information to the computer system 112 via one or more of the remote computers through input devices, such as a keyboard, a pointing device (commonly referred to as a mouse), a trackball, or a touch pad. Other input devices comprise microphones, satellite dishes, scanners, or the like. In addition to a monitor, the computer system 112 and/or remote computers might comprise other peripheral output devices, such as speakers and a printer.

Although many other internal components of the computer system 112 are not shown, such components and their interconnection are well known. Accordingly, additional details concerning the internal construction of the computer system 112 are not further disclosed herein.

Software components 116 may include a segmentation component 120, a thermal statistics tracking and update component 122, and a pattern recognition/classification component 124. One or more of such components may be used in tracking a position of the person within the facility 108 based on the thermal image data provided from each sensor 102 of the plurality of sensors 102. The skilled person will recognize that not all of such software components 116 need be used to perform such an analysis.

Generally, segmentation component 120 provides a segmentation algorithm to separate thermal image data of the person from background thermal image data of thermogram of the facility 108 provided from each sensor 102.

Further, generally, the thermal statistics tracking and update component 122 provides for the tracking of regional thermal statistics (e.g. thermal data representative of pixels in one or more of regions 110 for one or more regions of interest). In other words, this tracking and update component 122 performs a monitoring function which may update the regional thermal data dynamically from frame to frame grabbed by each sensor 102. In one example, the tracking and update component 122 can recognize when there is a change in the thermal data received from the sensors 102 in the facility 108, such as when a person enters the facility 108.

Lastly, in general, pattern recognition/classification component 124 may provide a pattern recognition algorithm operable upon thermal data representative of one or more of the regions 110. For example, pattern recognition/classification component 124 can provide for automatic classification of the person into regions 110. Preferably, the pattern recognition algorithm is an algorithm that is part of a class of algorithms using statistical learning methodology. This may be used to correct for some variability in the thermal signatures across the person of interest.

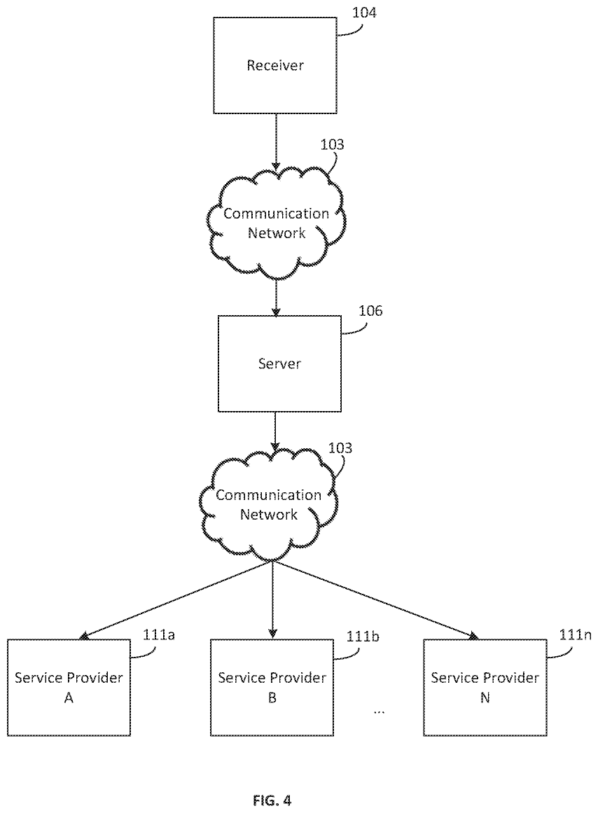

Referring now to FIG. 4, an optional embodiment is provided where server 106 is coupled to a plurality of service providers 111 (i.e. service providers 111a, 111b, . . . , 111n) via transmitter 126. In this embodiment, server 106 can provide each of the service providers 111 with data generated by software components 116. In one specific embodiment, the data that can be provided to the service providers 111 is a hygiene standards compliance profile. Examples of service providers 111 include but are not limited to hospitals, medical laboratory spaces, etc.

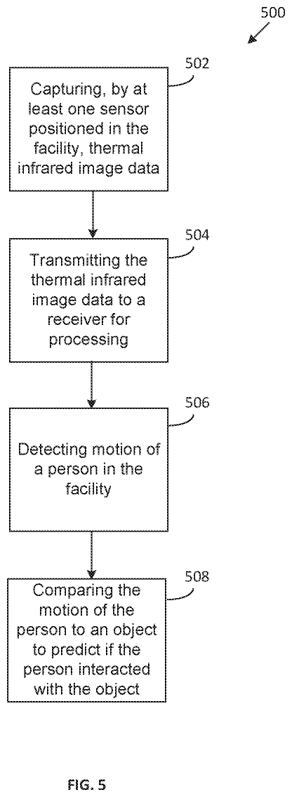

Referring now to FIG. 5, a method 500 of tracking a person of interest in a facility is provided.

At step 502, thermal infrared image data is captured by the one or more sensors 102. Typically, the sensors 102 will capture infrared image data within facility 108.

At step 504, the thermal infrared image data is transmitted to one of the receiver 104 and/or the server 106. In some embodiments, thermal infrared image data is transmitted from sensors 102 as a thermogram. In other embodiments, the thermal infrared image data is received by the receiver 104 and/or the server 106 and a thermogram is constructed. The thermogram indicates a position of a person in the facility 108.

At step 506, motion is detected. For example, in one embodiment, motion may be detected based on the image frames captured by sensors 102. In this regard, an appropriate motion detection process (e.g., an image registration process, a frame-to-frame difference calculation, or other appropriate process) may be applied to captured image frames to determine whether motion is present (e.g., whether static or moving image frames have been captured). For example, in one embodiment, it can be determined whether pixels or regions around the pixels of consecutive image frames have changed more than a user defined amount (e.g., a percentage and/or threshold value). If at least a given percentage of pixels have changed by at least the user defined amount, then motion will be detected.

At step 508, the motion of the person (e.g. the position of the person in the facility 108 over time) is compared to stored positions of objects in the facility 108 to determine if the person interacted with the objects in the facility 108. For instance, in some embodiments, the objects may be sanitation devices and the motion of the person may be compared to the positions of the sanitation devices to predict if the person utilized the sanitation devices. The positions of the sanitation devices can be stored in memory 118 of server 106.

Additionally, before the motion of the person is detected at step 506, thermal infrared person data may be isolated from the thermal infrared image data. For instance, isolating the thermal infrared person data may include performing edge detection on the thermal infrared image data to determine a portion of the thermal infrared image data that is thermal infrared person data. Thermal infrared person data generally corresponds to the position of at least one person in the facility 108. In some examples, local contrast values in the thermal infrared image data may be calculated, or any other desired type of edge detection process may be applied to identify certain pixels in the thermal infrared image data as being part of an area of local contrast.

Additionally, step 508 may further include determining a route of the person through the facility 108 based on the position of the person in the facility 108 over time.

Additionally, the method 500 may also include determining a likelihood that the person is a health care professional based on the route of the person through the facility.

While the above description provides examples of one or more apparatus, methods, or systems, it will be appreciated that other apparatus, methods, or systems may be within the scope of the claims as interpreted by one of skill in the art.

What is claimed is:

1. A method of tracking interactions of a person in a facility, the method comprising:
a) capturing, by at least one sensor positioned in the facility, thermal infrared image data;
b) transmitting the thermal infrared image data to a receiver for processing the thermal infrared image data;
c) detecting motion of at least one person in the facility by comparing pixels or regions around the pixels of consecutive image frames of thermal infrared image data received from the at least one sensor; and
d) when motion of at least one person in the facility is detected, comparing the motion of the at least one person in the facility to an object in the facility to predict if the person interacted with the object in the facility.

2. The method of claim 1, further comprising:
before the detecting motion of at least one person in the facility, isolating thermal infrared person data representing the position of the person in the facility from the thermal infrared image data.

3. The method of claim 2, wherein the isolating thermal infrared person data includes performing edge detection on the thermal infrared image data to identify the thermal infrared person data of the thermal infrared image data.

4. The method of claim 1, further comprising determining a route of the person through the facility based on the motion of the person in the facility over time.

5. The method of claim 4, further comprising determining a likelihood that the person is a health care professional based on the route of the person through the facility.

6. The method of claim 1, wherein the objects are personal hygiene devices, the at least one person in the facility is a health care worker, and the method further comprises:
creating a hygiene standards compliance profile based on determining if the health care worker interacted with the personal hygiene devices in the facility; and
forwarding the hygiene standards compliance profile to a third party service provider.

7. A system for tracking interactions of a person in a facility, the system comprising at least one processor configured to:
a) receive thermal infrared image data from at least one sensor positioned in the facility;
b) detect motion of at least one person in the facility by comparing pixels or regions around the pixels of consecutive image frames of thermal infrared image data received from the at least one sensor; and
c) when motion of at least one person in the facility is detected, compare the motion of the at least one person in the facility to an object in the facility to predict if the person interacted with the object in the facility.

8. The system of claim 7, wherein the processor is further configured to:
before detecting motion of at least one person in the facility, isolate thermal infrared person data representing the position of the person in the facility from the thermal infrared image data.

9. The system of claim 8, wherein the processor is further configured to:
isolate the thermal infrared person data by performing edge detection on the thermal infrared image data to identify the thermal infrared person data of the thermal infrared image data.

10. The system of claim 7, wherein the processor is further configured to:
determine a route of the person through the facility based on the motion of the person in the facility over time.

11. The system of claim 10, wherein the processor is further configured to:
determine a likelihood that the person is a health care professional based on the route of the person through the facility.

12. The system of claim 7, wherein the objects are personal hygiene devices, the at least one person in the facility is a health care worker, and the processor is further configured to:
create a hygiene standards compliance profile based on determining if the health care worker interacted with the personal hygiene devices in the facility; and
forward the hygiene standards compliance profile to a third party service provider.
